(19) United States
(12) Patent Application Publication
(10) Pub. No.: US 2006/0282135 A1
(43) Pub. Date: Dec. 14, 2006

(54) Radiation separating shield for skin treatment

(75) Inventor: Nikolai I. Tankovich, San Diego, CA (US)

Correspondence Address: JOHN R. ROSS, P.O. BOX 2138, DEL MAR, CA, 92014, US

(73) Assignee: STCMEDICA CELL TECHNOLOGIES, INC.

(21) Appl. No.: 11/395,811

(22) Filed: Mar. 31, 2006

Related U.S. Application Data
(63) Continuation-in-part of application No. 10/890,076, filed on Jul. 12, 2004.

(51) Int. Cl. A61N 5/06

(52) U.S. Cl. 607/89; 607/88

(57) ABSTRACT

A process utilizing a separating shield to produce tissue damage in small regions of skin without damage to neighboring tissue. This permits healthy physiological responses originating in the neighboring tissue to repair the damaged tissue or to produce new healthy tissue to replace the damaged tissue. In preferred embodiments a variety of separating shield materials are used to protect the neighboring tissues. And a wide variety of radiation sources may be utilized.

This invention relates to lasers and in particular to techniques relating to the use of lasers for skin treatments. This application is a continuation-in-part of Ser. No. 10/890,076 filed Jul. 12, 2004 which is incorporated herein by reference. This application also claims the benefit of Provisional Patent Application, Ser. No. 60/598,201 file Mar. 31, 2005.

### BACKGROUND OF THE INVENTION

Use of radiation for medical and cosmetic purposes is well known. When human tissue is damaged natural wound healing and immune responses from neighboring un-damaged cells produce a necessary proteins and cells to replace the damaged cells. The new tissue cells may be much healthier than the damaged cells were before they were damaged.

Lasers are used extensively for purposes such as hair removal, vein treatment, skin rejuvenation, treatment of telangeatesia and treatment of port wine stain. Each of these treatments is preferably preformed with a laser producing laser pulses at a wavelength chosen to be most effective for the particular treatment. For example, a Nd:YAG laser operating at 1064 nm may be used for hair removal and certain types of vein treatment. An Er:glass laser operating at 1540 may beused for skin rejuvenation and micro skin surgery. Treatent of port wine stain is usually performed using a dye laser operating at a wavelength of 577 nm. Lasers used for treatment of small surface veins do not work very well for treatment of larger deeper veins.

Some wavelengths are very preferentially absorbed in a particular type of tissue (referred to as a “chromophore”) that has a peak or relatively high absorption at the particular wavelength. Use of a laser beam matched to the peak or relatively high absorption in specific chromophores is referred to as “selective thermolysis”. For example the 532 nm wavelength of a frequency doubled YAG:Nd laser is highly absorbed by blood and is used to treat vascular lesions like telangeatesia and small facial veins. Some materials absorb relatively uniformly all wavelengths within broad spectral bands. Treatments directed at applying radiation energy to heat these materials are sometimes referred to as “non-selective thermolysis”. Some materials are highly reflective within specific spectral ranges which in some cases are narrow and in some cases the ranges may be very broad. For example metal particles as well as their oxides and salts tend to reflect light very well in the visible and infrared spectral ranges.

What is needed is a technique for producing damage is small sections of tissue while leaving surrounding tissue healthy.

### SUMMARY OF THE INVENTION

The present invention provides a process utilizing a separating shield to produce tissue damage in small regions of skin without damage to neighboring tissue. This permits healthy physiological responses originating in the neighboring tissue to repair the damaged tissue or to produce new healthy tissue to replace the damaged tissue. In preferred embodiments a variety of separating shield materials are used to protect the neighboring tissues. And a wide variety of radiation sources may be utilized.

### BRIEF DESCRIPTION OF THE DRAWINGS

FIG. 1A is a drawing of a human skin in cross section.

FIG. 1B is a drawing showing a skin shielding lotion containing suspended light absorbing particles.

FIG. 2 is a drawing showing a skin gel moist pad with suspended light absorbing particles used to protect portions of the skin from effects of light radiation.

FIG. 3 shows an oil solution with suspended light reflecting particles used to protect portions of the skin from effects of light radiation.

FIG. 4 shows an oil solution with suspended light scattering particles used to minimize the effects of light radiation on portions of the skin.

FIG. 5 shows the focusing effects of a particular shielding skin lotion.

FIG. 6 shows the effects of adding Fulerin particles to the lotion shown in FIG. 5.

FIG. 7 shows a shielding lotion used in a hair removal procedure.

FIGS. 8A and 8B shown a grid of carbon threads used to protect portions of a skin region from damage from light radiation.

FIG. 9 shows a wrinkle removal procedure.

FIG. 10 shows a procedure for treating stretch marks.

FIG. 11 shows a shield used to protect a neoplastic lesion from light radiation.

FIG. 12 shows a process for driving a bleaching compound into a benign pigmented lesion.

FIGS. 13A, 13B and 13C show techniques for using a mask to producing skin damage in patterns of tiny spots.

FIG. 14 shows a technique for producing a pattern of skin damage by dipping a laser hand-piece into a particle containing sticky fluid.

FIG. 15 shows a sunscreen lotion used on the skin to permit some skin damage but protect surrounding tissue to permit quick rejuvenation.

FIG.16 shows the use of carbon particles as a skin lotion to absorb long wavelength light and to transfer the captured heat energy to skin tissue.

FIG. 17 shows a shielding lotion used to shield skin surrounding an acne eruption being radiated.

FIG. 18 shows the use of light absorbing particles and a short pulse laser to drive photo-dynamic therapy drugs into the skin tissue.

FIG. 19 is a drawing (FIG. 13) from the parent application Ser. No. 10/890,076 showing elements of the present invention.

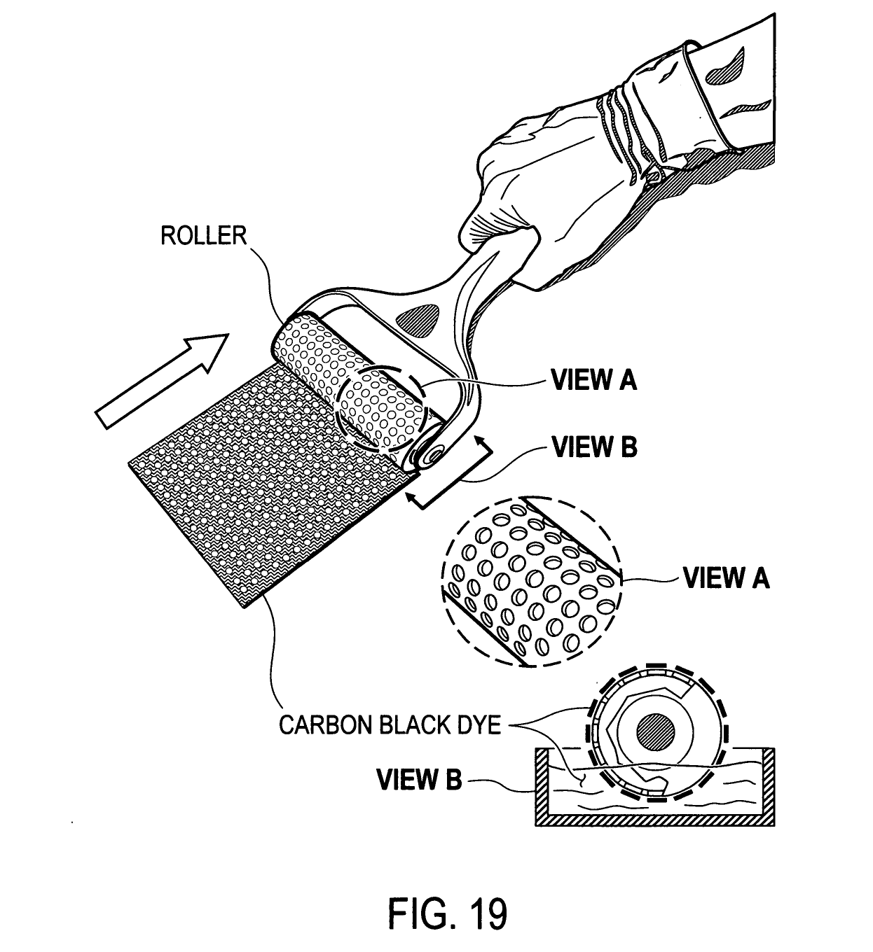

### DETAILED DESCRIPTION OF PREFERRED EMBODIMENTS

### Description from Parent Patent Application

The basic concept of the present invention was described in the parent patent application listed in the first sentence of this specification. This is the concept of protecting some tissue in radiation treated skin regions so that natural immune responses provided by the protected healthy tissue will result in quick and effective healing of skin tissue damaged by the radiation treatment. FIG. 20 is a drawing from the parent application, Ser. No. 10/890,076 filed Jul. 12, 2004, showing elements of the present invention. As stated in that application, “Masking the skin surface preserves healthy portions of the skin for a fast normal healing process”. Immune and wound healing response comes from living tissues and cells. If all the cells in a skin region are dead then the physiological response must come from outside the region. If large regions are damaged in laser treatments, without leaving healthy tissue in the region, healing will generally take much longer and the healing process generally will not be nearly as successful as when some undamaged healthy tissue is left in the region.

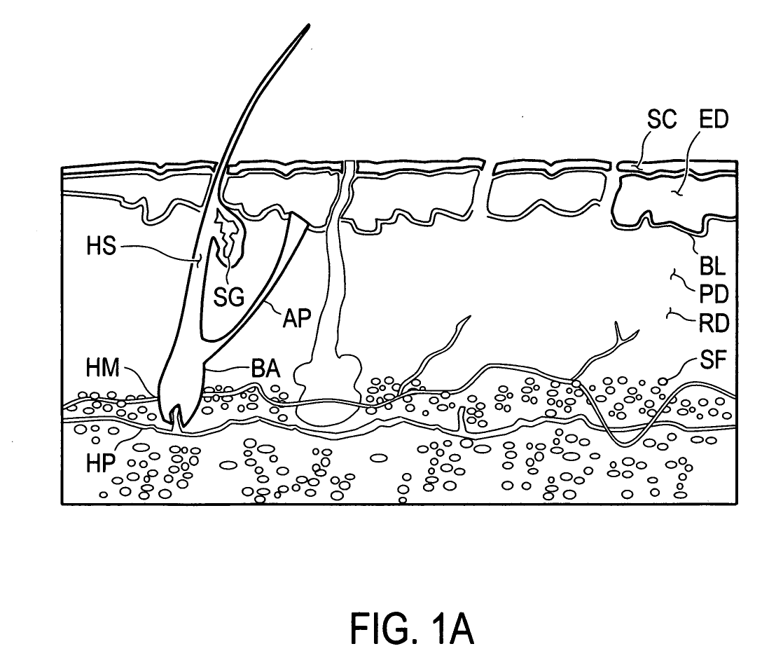

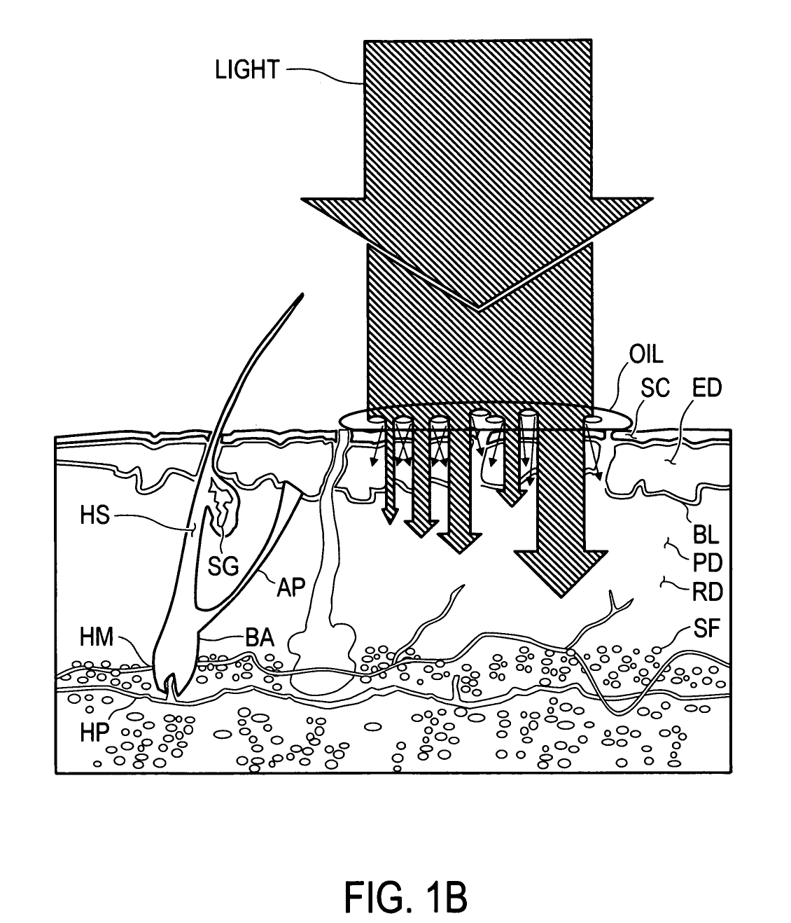

FIG. 1A is a drawing of a human skin in cross section in a region about two millimeters thick. The epidermis contains five major layers or stratums called (from the top down) corneum, lucidum, granulosum, spinosum and basale. The epidermis is about 100 microns thick. Below the epidermis is the dermis that consists of two layers the papillary and the recticular dermis. These and other features of the skin shown in FIG. 1A are the SC, the epidermis ED, the basal layer of the epidermis BL, the dermal papilla DP, the recticular dermis RD, the subcutaneous fat SC, the sebaceous gland SG, a hair shaft HS, the arrector pili muscle AP, a bulge area hair follicle BA, a hair bulb with matrix of hair bulb HB and a blood vessels of hair papilla HP. The thickness of the dermis varies from about 3 to 6 millimeters. These are the skin features that are damaged to various degrees when the skin is radiated with damaging radiation which as indicated above could be any of a wide range of radiation from high intensity radiation (in the radio wave to visible light portion of the spectrum) to single photons of gamma, x-ray or ultraviolet radiation. These are also the features that must be healed or replaced after the damage is done.

### Techniques for Leaving Healthy Skin Tissue in Radiation Treated Skin Regions

As indicated in the Background Section of this specification, human skin is treated with a wide range of radiation, from including radio wave, microwave, infrared light, visible light, and ultraviolet light and even x-ray and gamma ray radiation. Most skin treatments, using radiation, produce damage to skin tissue and the treatment relies on the patient's normal wound healing and immune processes to react and produce repair and/or replacement of some or all of the tissue. In some cases, like hair removal the idea is to produce permanent damage to some tissue but to minimize damage to surrounding tissue. In other cases like skin rejuvenation the objective is to avoid permanent damage or to minimize it as much as possible. There are many available techniques for producing damage to regions while leaving healthy tissue in neighboring regions. Many to these techniques are described in the figures and in the following sections of this specification:

Scattering Particles Suspended in Lotion

FIG. 1B is a drawing showing a skin shielding lotion containing suspended light scattering particles. The shielding could be any one of many lotions transparent to the radiation. And the particles can be any of a large variety of particles that scatter the radiation. An example would be laser light at from a Er:Glass diode pump system at a wavelength of 1.5 micron with baby oil as the lotion and Titanium dioxide as the suspended particles. The number and size of the particles are chosen so that some skin tissue is in line with and protected in part by the scattering particles and some tissue is not in line with and receive substantially the full illumination flux plus some of the flux scattered from the particles. As shown in the drawings the particles scatter light that illuminate the particles reducing the flux below the particles while light that does not illuminate the particles pass through to the skin at approximately full intensity so that tissue not in line with the particles receive substantially more than the average flux and protected (inline) tissue substantially less than the average flux. Persons skilled in the art will be aware of many other combinations of radiation, lotions and scattering that can be utilized for skin treatments using the concept shown in FIG. 1B.

Impregnated Pad Shield

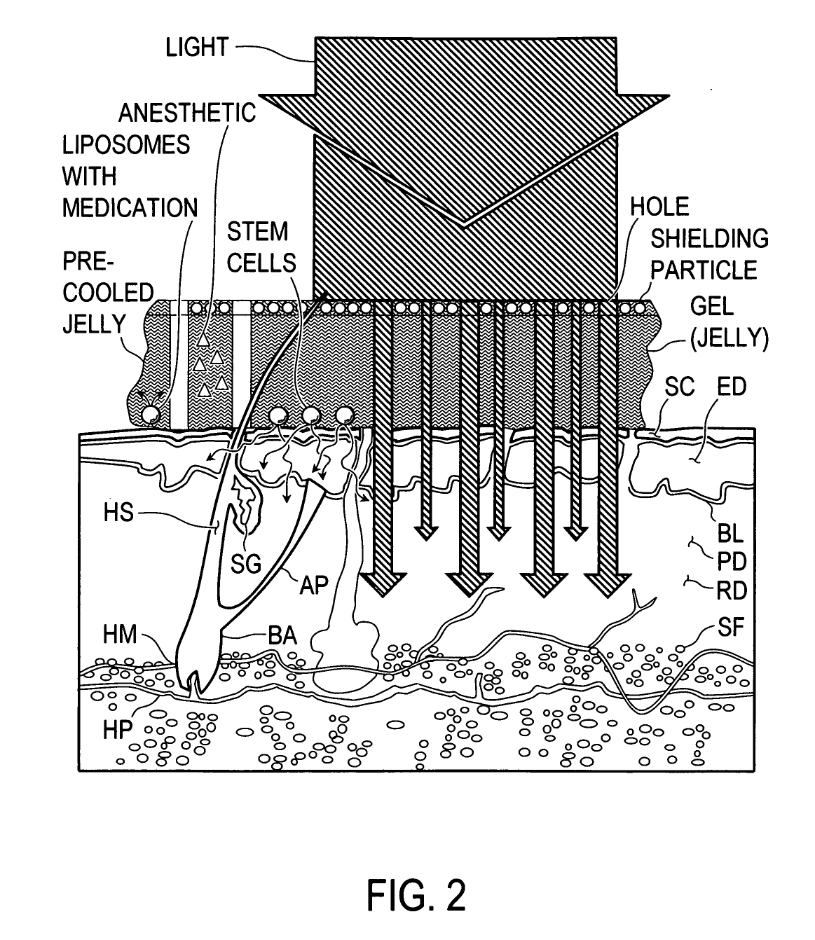

FIG. 2 is a drawing showing a skin gel moist pad with suspended light absorbing particles used to protect portions of the skin from effects of light radiation. Applicants utilized a special process to imbed carbon particles in the skin gel moist pad. The pad is transparent Second Skin Gel Moist Pad available from Spenco Medical Corporation with offices in Waco, Texas. Applicants imbedded the carbon particles by covering the pad thin layer of 20 percent graphite (one micron size) particles in baby oil. The graphite-baby oil mixture was then radiated with short pulse laser beams from a Q-switched Nd:YAG laser that explodes the one micron graphite particles and embeds smaller graphite particles in the pad. In preferred embodiments the embedded particles cover about sixty percent of the area of the pad. However, this percentage could be any percentage from near zero to nearly one hundred percent. The pad as supplied contains and anesthetic and preferably is pre-cooled before use for skin treatment. The embedded graphite particles are shown at the top of the pad in FIG. 2. The pad is then used for skin treatment with a light beam which may be any of many light beams including beams produced by the same Q-switched Nd:YAG that is used to embed the graphite particles. As shown in FIG. 2 some portions of the skin is protected by the graphite particles and other portions receive substantially the full laser flux. The pre-cooled pad protects the tissue very close to the skin surface from heat damage while the underlying tissue can be damaged by virtue of higher temperatures. Liposomes and/or medications can be applied at the bottom of the pad as shown in FIG. 2 and driven into the skin as a consequence of reactions with the laser radiation.

Light Reflecting Particles in Baby Oil

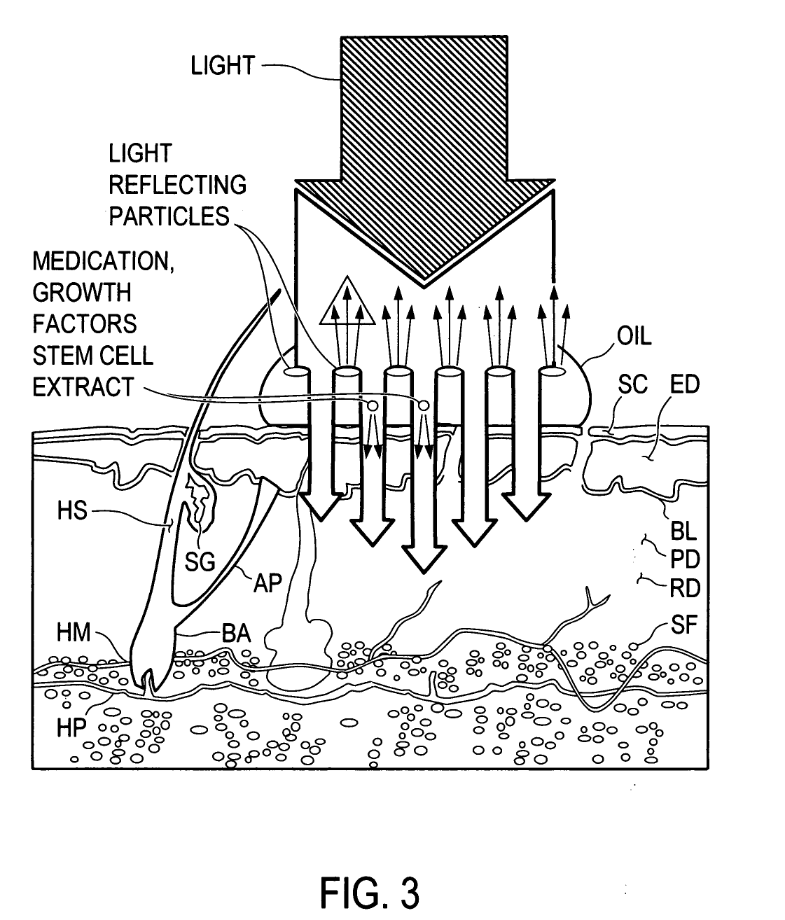

FIG. 3 shows a baby oil solution with suspended light reflecting particles used to protect portions of the skin from effects of light radiation. The light reflecting particles in a preferred embodiment are preferably aluminum flakes about 10 microns wide and about 1 micron thick. These flakes are available from Air Touch SK-II made by P&G. The light reflecting particles could be suspended in any of a very large number of transparent viscous fluids in addition to baby oil. In a similar embodiment (not shown) the reflecting flakes are attached to the top of a skin gel moist pad of the type described in the above section. Skin tissue in line with the light reflecting particles is mostly protected and is not substantially damaged by the radiation while skin tissue not protected receives far more radiation (including straight through radiation plus some radiation reflected off the particles) and is damaged.

Light Scattering Particles in Baby Oil

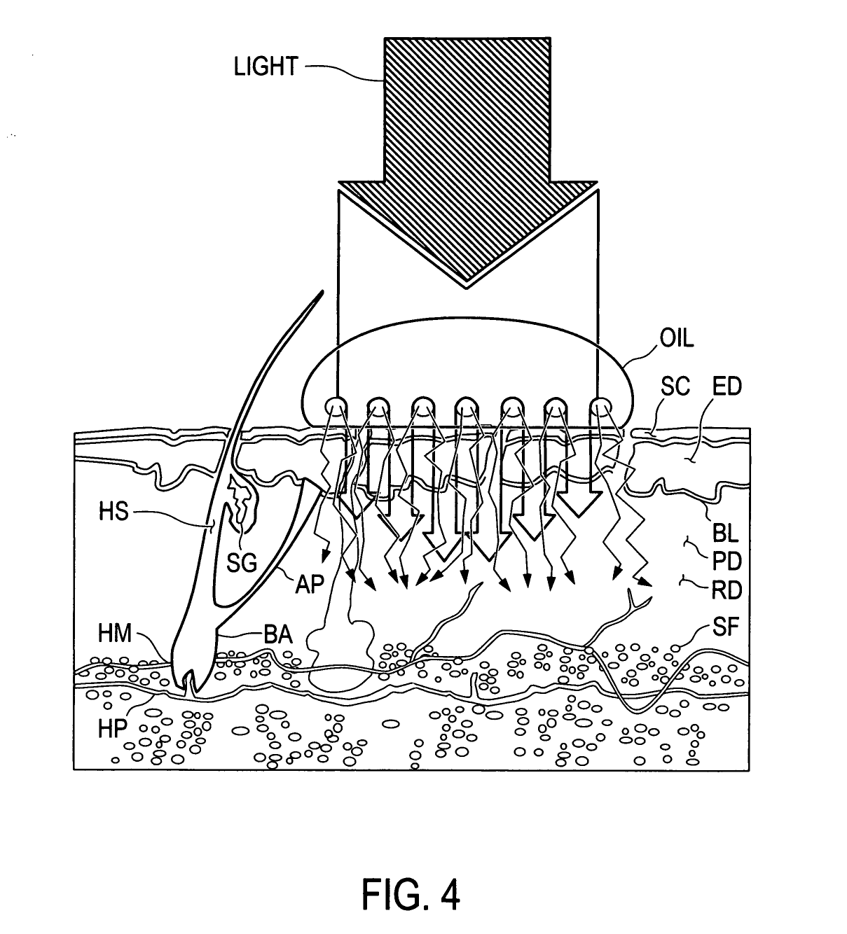

FIG. 4 shows an oil solution with suspended light scattering particles used to minimize the effects of light radiation on portions of the skin. Embodiments of this type are very similar to the embodiments described in the above section except the particles are light scattering particles rather than light reflecting particles. Some examples of good particles for use in these embodiments are latex micro-balls and coated polymer micro-balls.

Light Focusing Skin Elements

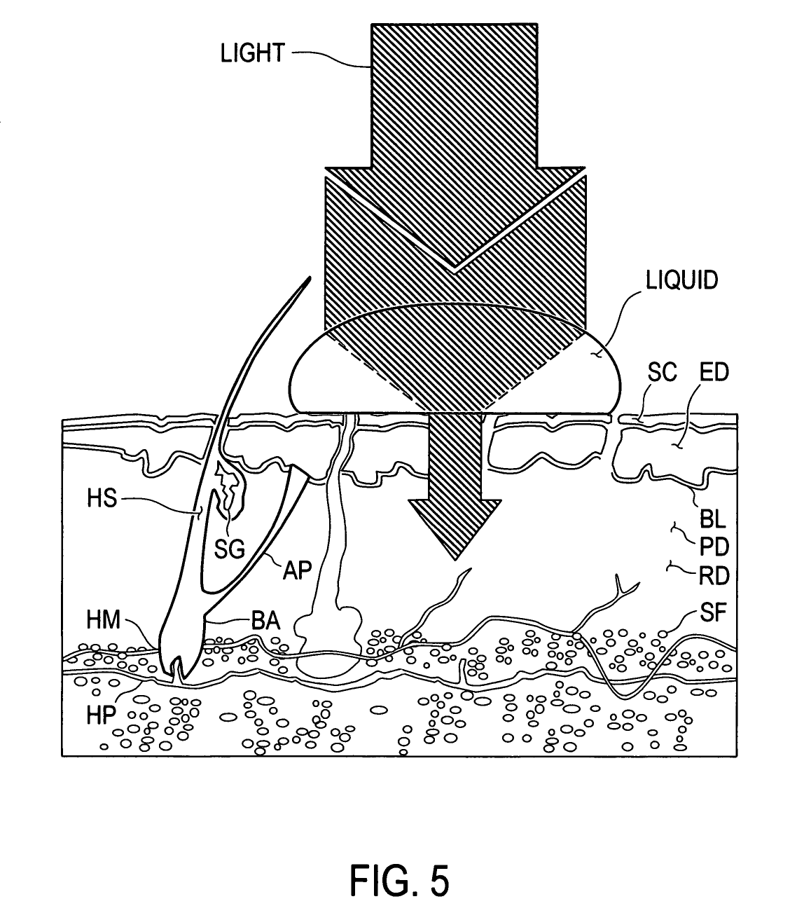

FIG. 5 shows the focusing effects of a particular shielding skin lotion. In this embodiment a liquid is chosen that is transparent to the source of skin illumination and forms bubbles when applied to the skin surface. Examples of liquids that have these properties are glycerin, tea tree oil, and KY gel. These liquids all have indexes of refraction greater than air and will therefore focus the illuminating laser light to only a portion of the region of the bubble as shown in FIG. 5. Therefore, portions of the skin are damaged and portions are not.

Addition of Light Absorbing to Focusing Elements

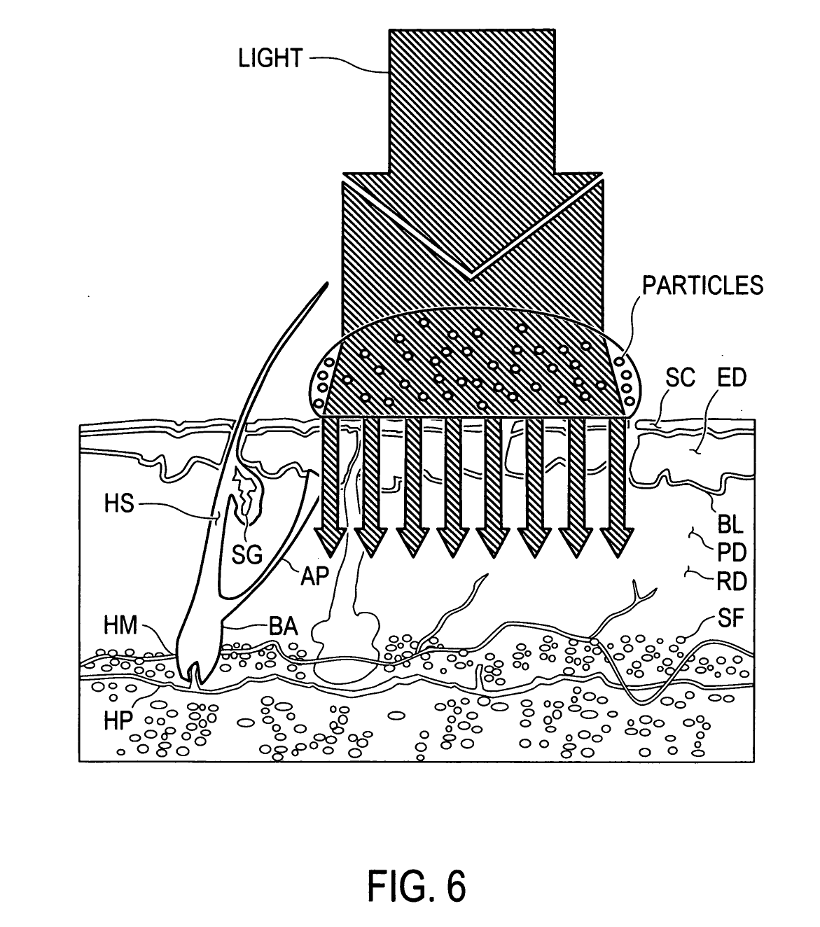

FIG. 6 shows the effects of adding Fulerin particles to the lotion shown in FIG. 5. Fulerin particles are very absorptive of almost all laser radiation. So skin regions in line with the particles will be protected and not damaged nearly to the extent of tissue not in line.

Hair Removal

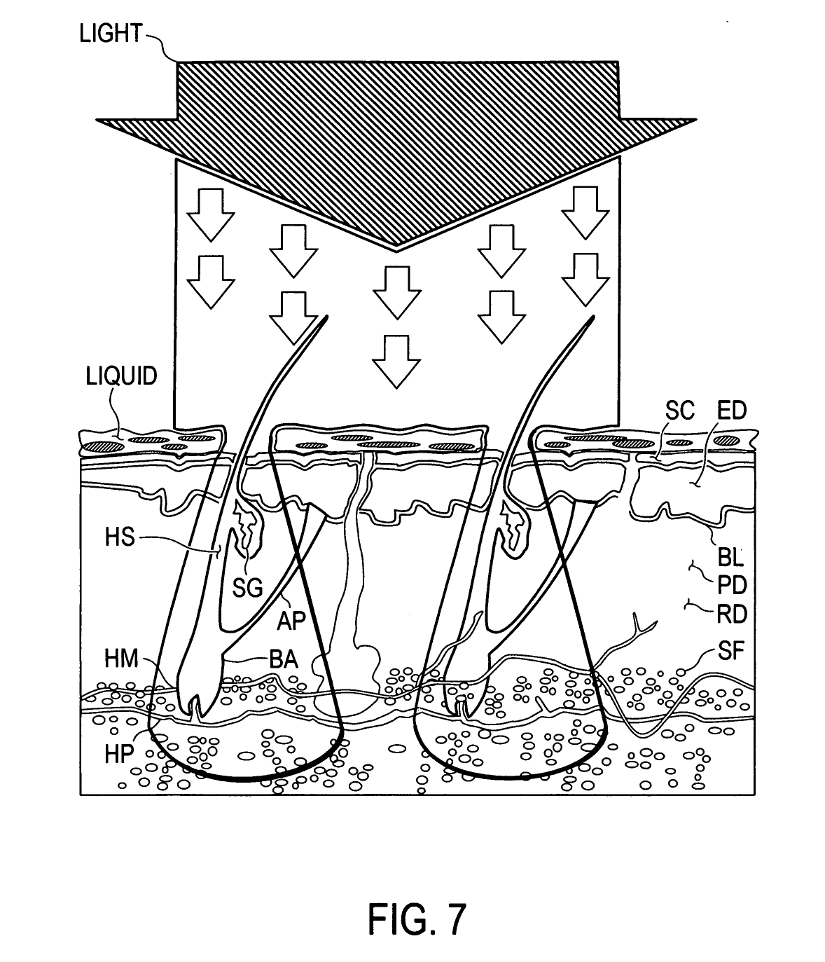

FIG. 7 shows a shielding lotion comprised of twenty percent graphite particles in baby oil cover a section of skin and used in a hair removal procedure. A laser path is provided where the hair shafts extend through the solution. The laser beam is prevented from reaching the skin except where the hair shafts are located. Thus, the skin is damaged only where the hair is located. Skin tissue surrounding the hair shaft will replace the damaged tissue with scar tissue but without the hair elements.

Grid Mask

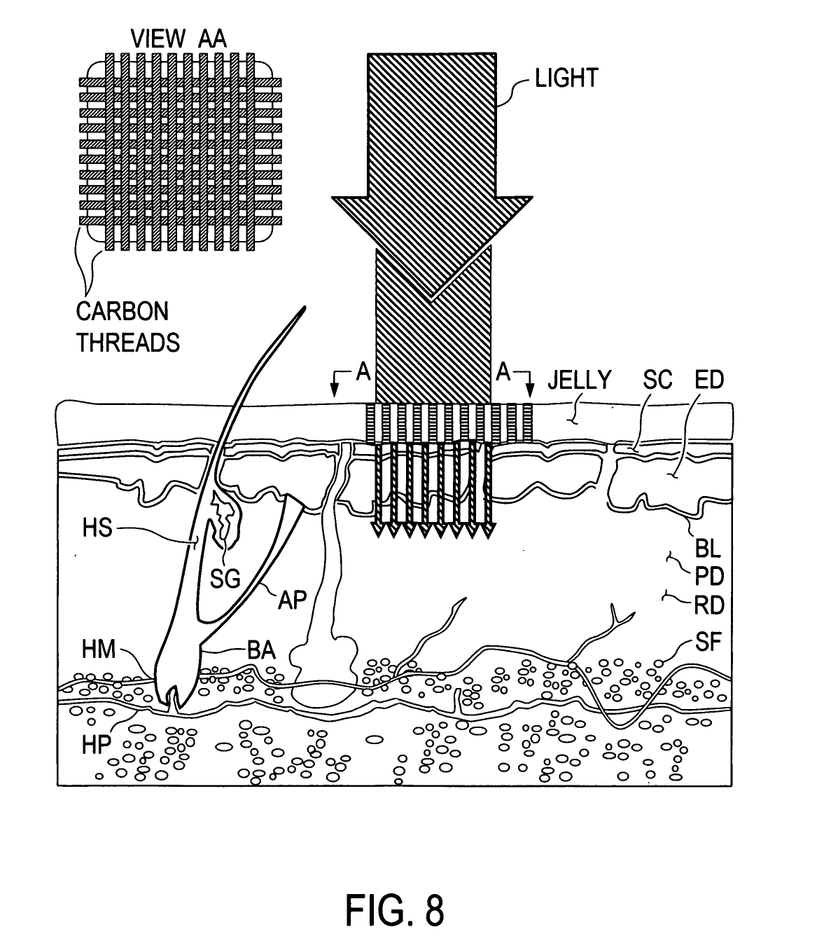

FIGS. 8A and 8B shown a grid of carbon threads used to protect portions of a skin region from damage from light radiation. In a preferred embodiment the threads are about 100 microns thick and spaced on 400 micron centers. As above regions of the skin are damaged and neighboring regions not damaged provide the immune responses to heal or replace the damaged tissue.

Wrinkle Removal

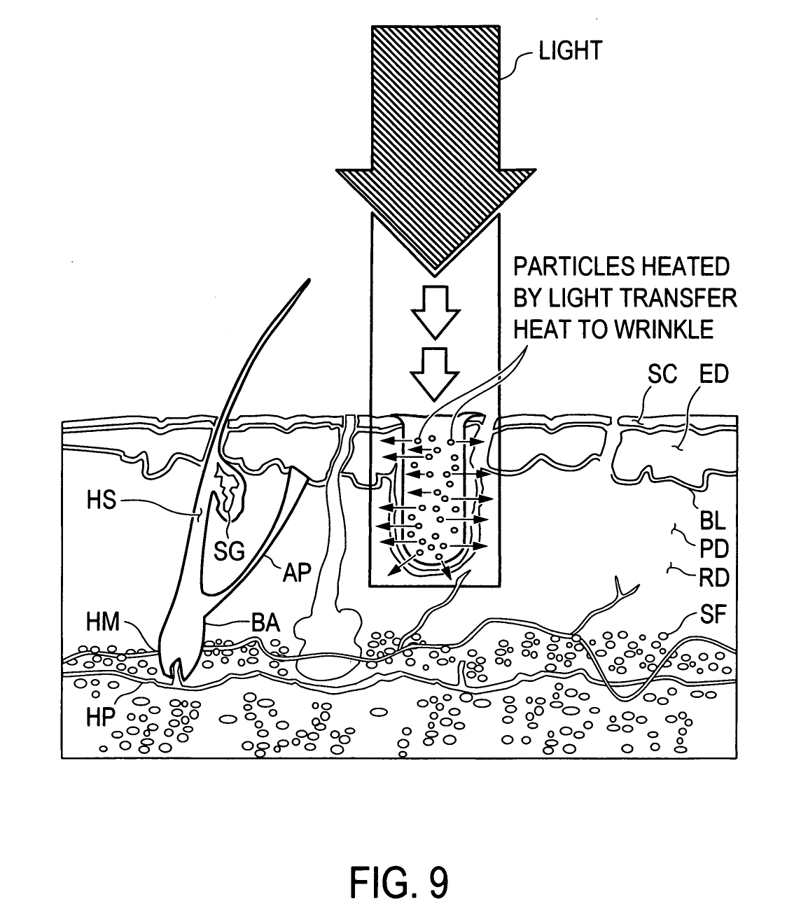

FIG. 9 shows a wrinkle removal procedure. Here a twenty percent graphite in baby oil solution is rubbed on the skin then the skin is wiped clean except for parts of the solution left in wrinkles on the skin. The skin surface is then lased with a laser wavelength that scatters well in skin tissue such as the 100 micron beam from the 1.5 micron laser. Virtually all the laser flux illuminating into or scattering into the graphite-filled wrinkle will be absorbed heating the graphite to a very high temperature that damages the tissue in the immediate region of the wrinkle. Surrounding tissue is not damaged and is available to provide the immune features for replacing the tissue in the wrinkle region.

Stretch Mark Removal

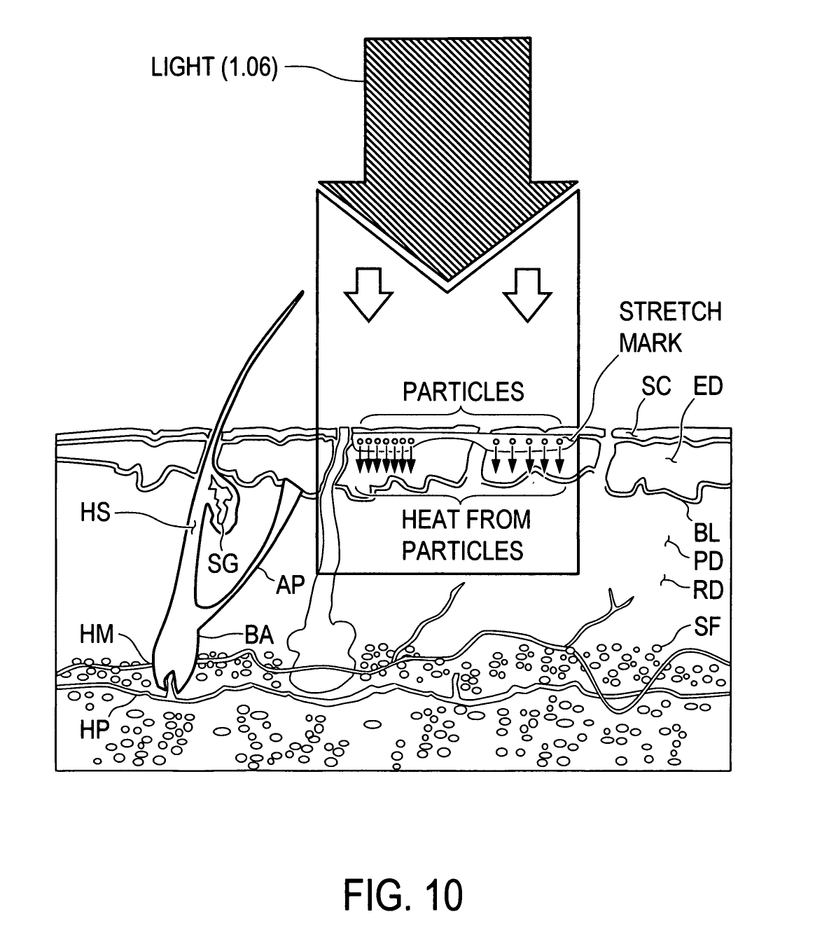

FIG. 10 shows a procedure for treating stretch marks. This process is basically the same as the one described above except the carbon particle-baby oil solution is concentrated in stretch marks rather than wrinkles.

Neoplasia Protection

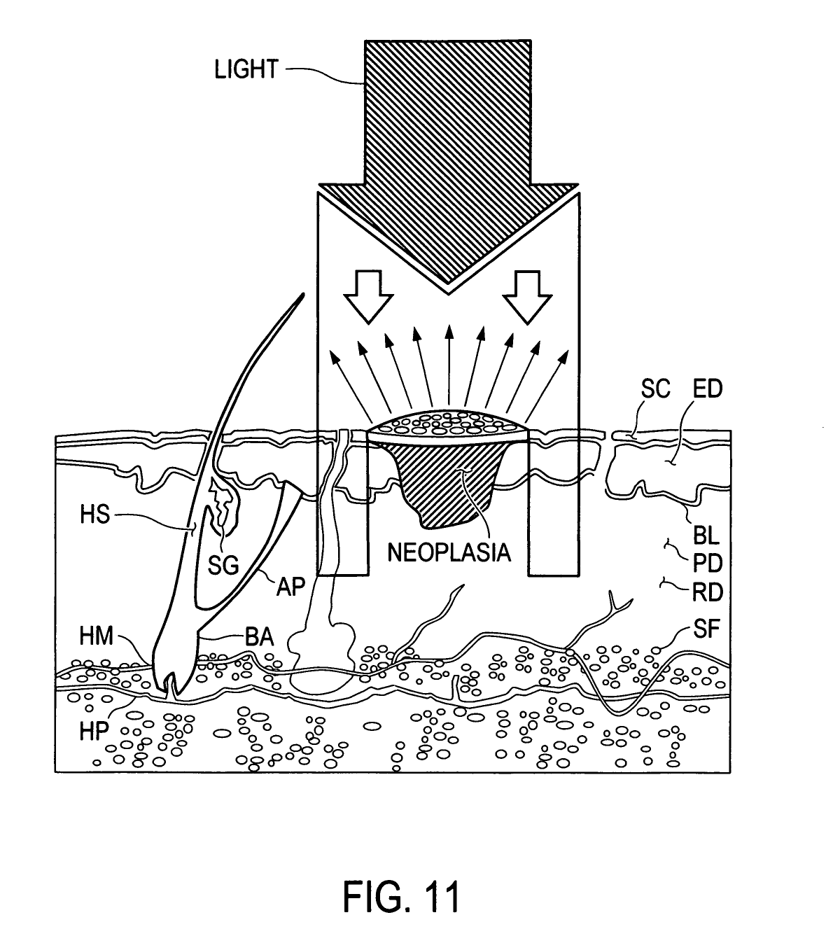

FIG. 11 shows a shield used to protect a neoplastic lesion from light radiation. A neoplastic lesion on the skin is a new abnormal growth. It may be cancerous and it may be nothing to worry about. What we do not want to do is encourage its growth. A possible treatment is to produce some damage to skin immediately surrounding it so that its growth is discouraged, but to leave undamaged tissue surrounding the surrounding tissue to assure recovery of the surrounding tissue. In the case shown in FIG. 11 the neoplastic lesion is coated with a shield of reflecting particles in solution so that the neoplastic lesion tissue is not damaged but the immediate surrounding tissue is. Tissue surrounding that tissue is not irradiated so it is also not damaged.

Bleaching Compound for Benign Pigmented Lesion

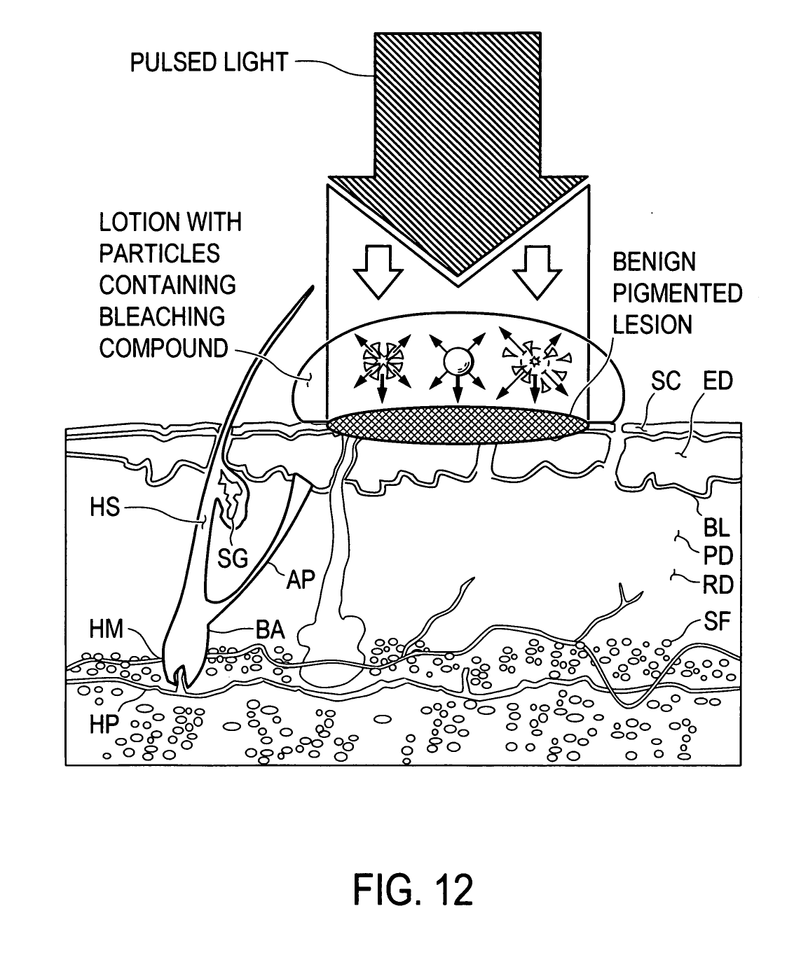

FIG. 12 shows a process for driving a bleaching compound into a benign pigmented lesion. Often people will develop pigmented skin regions. Chemicals are available that can be rubbed into these regions that will bleach out the unwanted pigment. The chemicals are more effective if inserted into the skin. In the FIG. 12 example the chemical (such as hydroquinone) is contained within carbon particles that are mixed with baby oil and coated over the pigmented lesion. The coating is then lased with a short pulse laser beam that explodes the carbon particle driving the bleaching agent into the pigmented lesion. Any tissue damaged is repaired by surrounding tissue not damaged.

Use of Masks to Produce Tiny Damaged Spots

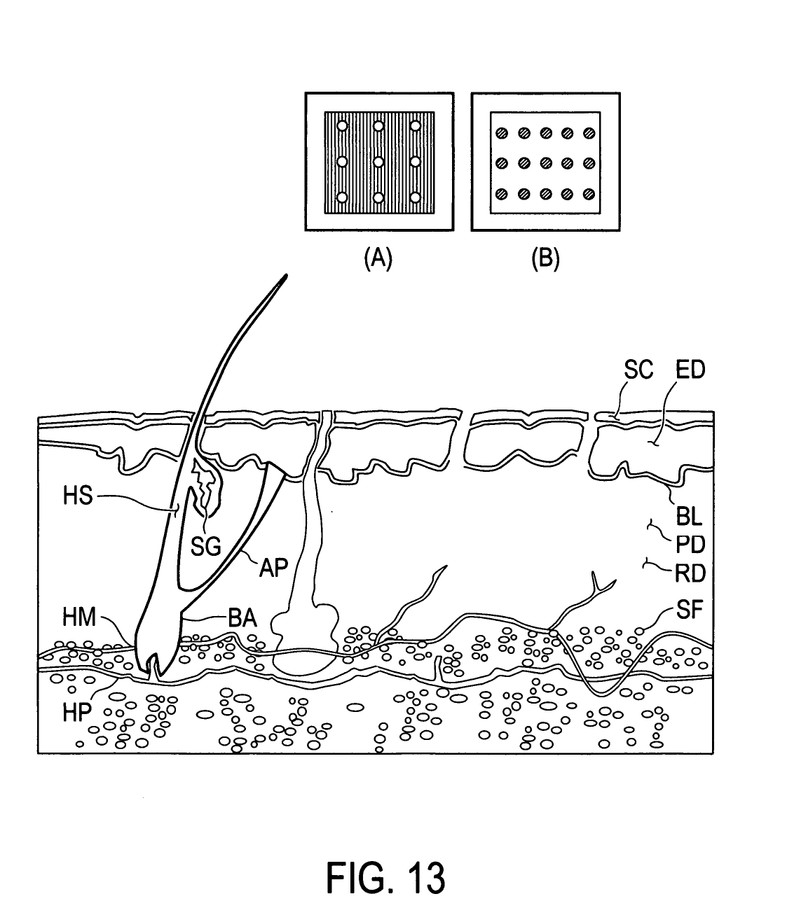

FIGS. 13A, 13B and 13C show techniques for using a mask to producing skin damage in patterns of tiny spots. The mask can be made of an absorbing material as in FIG. 13A or a reflecting material as in FIG. 13B. In either case when used on a skin section as in FIG. 13, the only skin damage is where the holes are located. The rest of the skin is not damaged. Stem cells or growth factors can be applied to the region and they will be most effective only in the small spots where the skin is damaged. The surrounding skin regions will help in the healing as described above.

Particle Containing Sticky Fluid

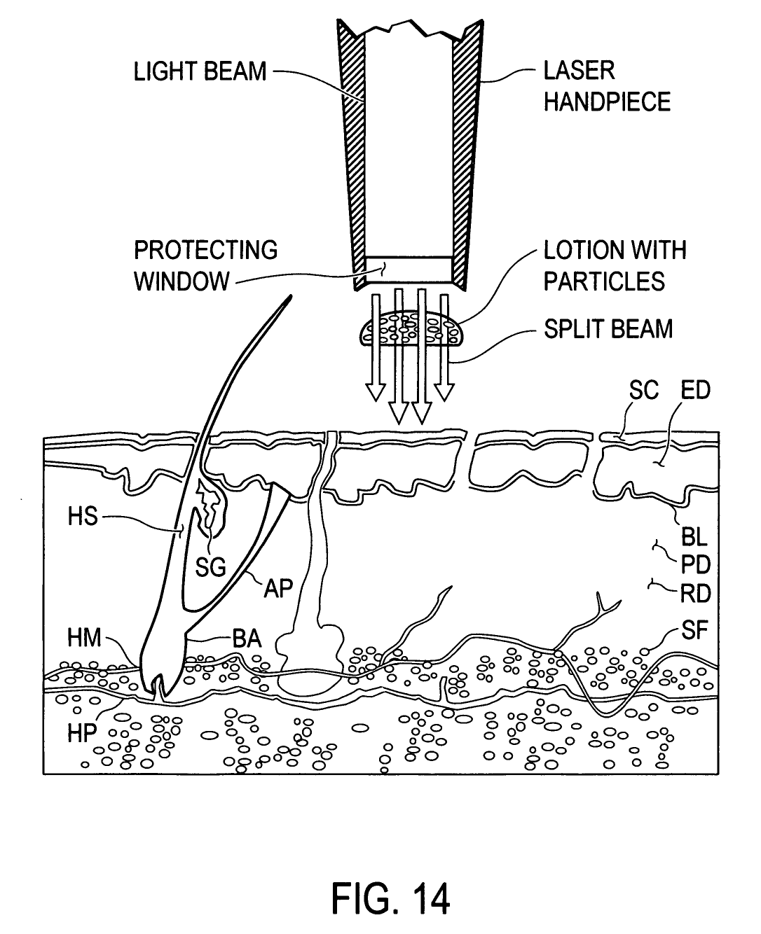

FIG. 14 shows an easy to use technique for producing a pattern of skin damage by dipping a laser hand-piece into a particle containing sticky fluid. The sticky fluid is transparent to the laser light but the particles are very absorptive or very reflective. The size and the concentration of the particles should be adjusted to achieve the desired results. As in the above examples some regions of the skin will be damaged and some regions will be protected.

Intentional Sunburns

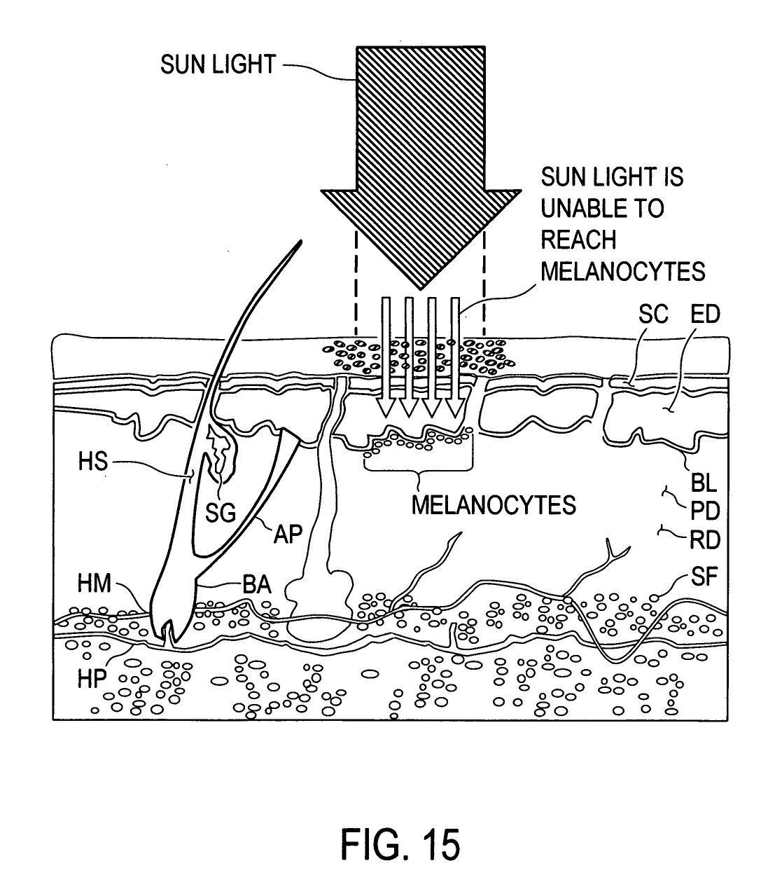

FIG. 15 shows a sunscreen lotion used on the skin to permit some skin damage but protect surrounding tissue to permit quick rejuvenation. FIG. 15 demonstrates how this invention can be used to provide skin rejuvenation via intentional sunburns. It is well known that sunburns often produce blisters that result in pealing of the skin. The skin that peals off is replaced by new skin and the person thus has new younger skin. This invention provides a technique to use the sun to rejuvenate your skin without the need to go through the blistering and pealing process. In a preferred embodiment a sunscreen lotion is prepared as a mixture of five micron graphite particles in baby oil (10 percent graphite by volume). It is applied to the skin in a layer thick enough so that about one half of the skin surface is covered by the graphite. The user should experiment with gradually increasing the exposure time so as to not receive excessive burns.

Hot Separated Radiation Absorbers

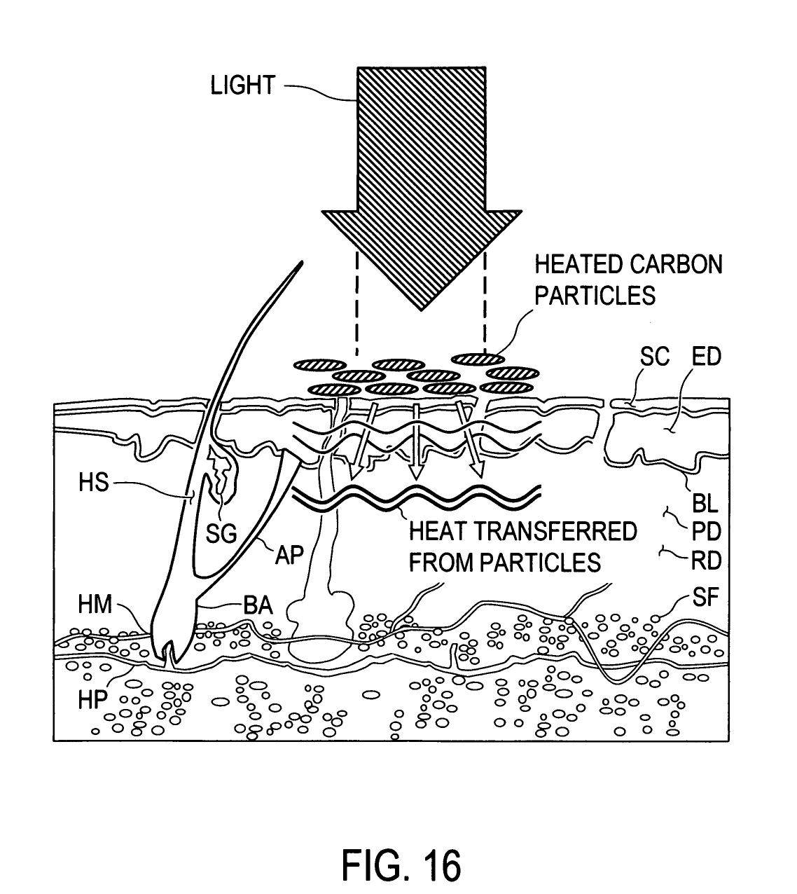

FIG. 16 shows the use of carbon particles as a skin lotion to absorb long wavelength light and to transfer the captured heat energy to skin tissue. With this technique light at any wavelength can be used including visible light, infrared, microwave and radio waves. The energy is absorbed in large energy absorbing particles such as graphite. Preferably the particles are about one or two millimeters and are mixed into an oil such as baby oil. The particles contacting the skin are separated by one or two millimeters so that as in the above embodiments only isolated portions of the skin are damaged leaving neighboring tissue undamaged.

Acne Treatment

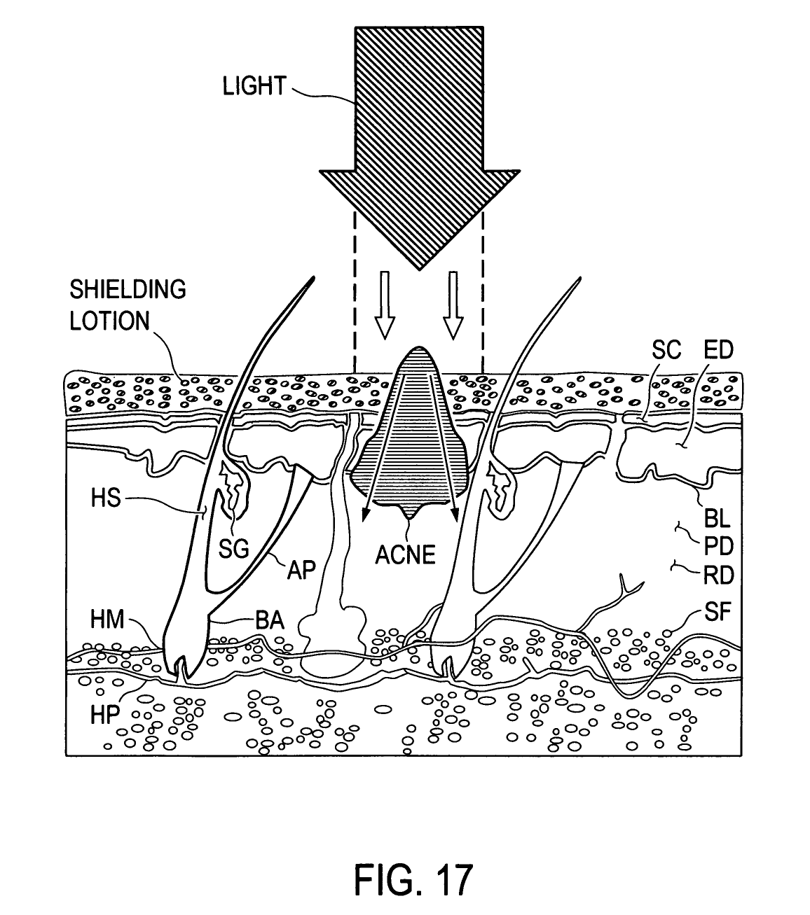

FIG. 17 shows a shielding lotion used to shield skin surrounding an acne eruption being radiated. The skin around the eruption is protected from damage while the acne region suffers damage curing the eruption and the damaged tissue is repaired or replaced be immune responses from that surrounding tissue.

Photodynamic Therapy

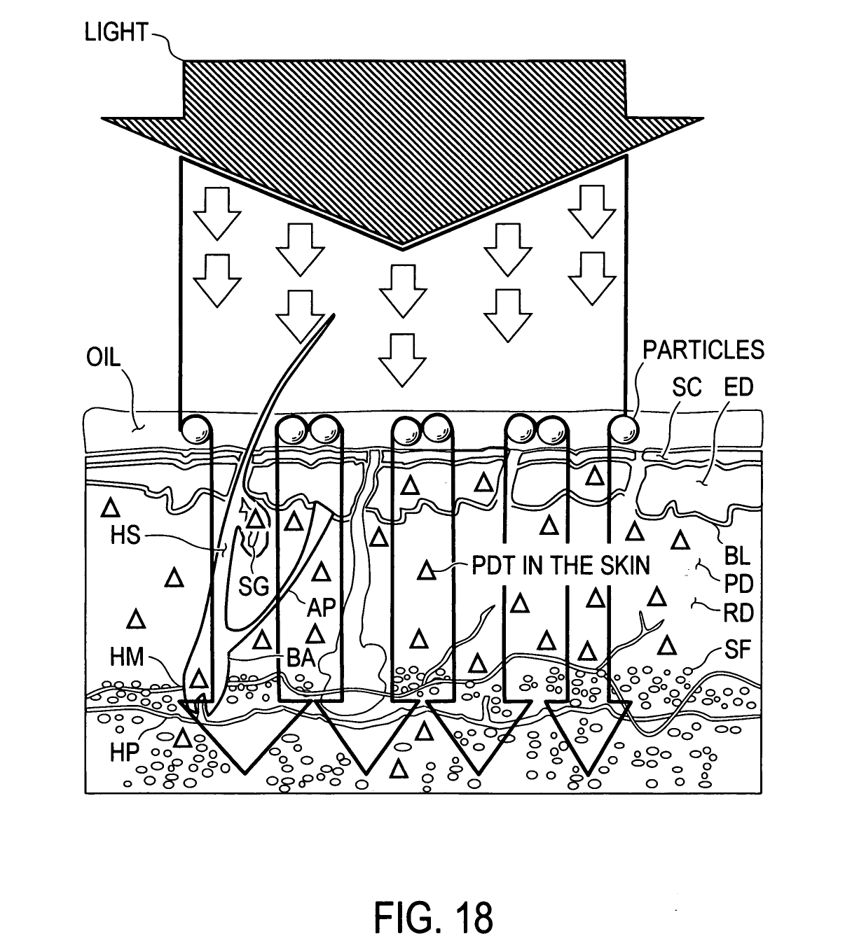

Photodynamic therapy is a treatment that uses a drug, called a photo-sensitizer or photosensitizing agent, and a particular type of light. When photo-sensitizers are exposed to a specific wavelength of light, they produce a form of oxygen that kills nearby cells. FIG. 18 shows the use of light absorbing particles to protect a portion of the skin during photodynamic therapy treatment. The photodynamic therapy drug may be administered in orally or intravenously. Relatively large (about 1 to 3 mm) graphite particles are mixed in baby oil, about 10 percent graphite by volume. The mixture is spread on the skin in a layer thick enough so that part of the skin is shaded by the particles and part is not shaded. The portion shaded may be any amount from near zero to near 100 percent. A good for many treatments is about 50 percent as shown in FIG. 18.

While the present invention has been described above in terms of specific preferred embodiments, persons skilled in the art will recognize that many modifications of these specific embodiments are possible without departing from the basic concepts of the present invention. Therefore, the reader should determine the scope of the present invention by the appended claims and their legal equivalents.

What is claimed is:

1. A process for producing tissue damage with radiation in small regions without damage to neighboring tissue comprising the steps of:
A) applying between skin of a person and a radiation source a radiation separating shield to separate the radiation passing through the shield to produce damage to skin tissue in small regions of the skin to produce a plurality of damaged regions while protecting from damage other regions of the skin that neighbor the damaged regions and
B) allowing the persons natural immune responses originating in the regions of the skin protected from damage to repair and/or replace skin tissue damaged in the damaged region.

2. The process as in claim 1 wherein the radiation source is a radiation source chosen from a group of sources consisting of the following sources: radio waves, microwaves, infrared light, visible light, and ultraviolet light and even x-ray and gamma ray radiation.

3. The process as in claim 1 wherein said separating shield is a skin shielding lotion containing suspended light absorbing particles.

4. The process as in claim 1 wherein said separating shield is a skin gel moist pad with suspended light absorbing particles used to protect portions of the skin from effects of light radiation.

5. The process as in claim 1 wherein said separating shield is an oil solution with suspended light reflecting particles used to protect portions of the skin from effects of light radiation.

6. The process as in claim 1 wherein said separating shield is an oil solution with suspended light scattering particles used to minimize the effects of light radiation on portions of the skin.

7. The process as in claim 1 wherein said separating shield comprises focusing elements to focus radiation to small regions of the skin.

8. The process as in claim 1 wherein said separating shield containing Fullerine particles.

9. The process as in claim 1 wherein said separating shield is used in a hair removal procedure.

10. The process as in claim 1 wherein said separating shield comprises a grid of carbon threads used to protect portions of a skin region from damage from light radiation.

11. The process as in claim 1 wherein said separating shield is comprised of radiation absorbing elements disposed in wrinkles in the skin in a wrinkle removal procedure.

12. The process as in claim 1 wherein said separating shield is comprised of radiation absorbing elements disposed in stretch marks in the skin in a stretch mark removal procedure.

13. The process as in claim 1 wherein said separating shield is used to protect a neoplastic lesion from light radiation.

14. The process as in claim 1 wherein said separating shield is utilized in a process for driving a bleaching compound into a benign pigmented lesion.

15. The process as in claim 1 wherein said separating shield is a mask to producing skin damage in patterns of tiny spots.

16. The process as in claim 1 wherein said separating shield is produced by dipping a laser hand-piece into a particle containing sticky fluid.

17. The process as in claim 1 wherein said radiation source is the sun and said separating shield is a sunscreen lotion used on the skin to permit some skin damage but protect surrounding tissue to permit quick rejuvenation.

18. The process as in claim 1 wherein said radiation source is the a skin tanning device and said separating shield is a sunscreen lotion used on the skin to permit some skin damage but protect surrounding tissue to permit quick rejuvenation.

19. The process as in claim 1 wherein said separating shield comprises radiation absorbing particles in a skin lotion for absorbing light to transfer the captured heat energy to skin tissue.

20. The process as in claim 1 wherein said separating shield is used to protect skin surrounding an acne eruption being radiated.

21. The process as in claim 1 wherein said separating shield is used to protect skin sections when the person has been treated with photo-dynamic therapy drugs.

22. The process as in claim 1 wherein said separating shield is a skin shielding lotion containing medication.
